(19) United States
(12) Patent Application Publication
(10) Pub. No.: US 2009/0196861 A1
(43) Pub. Date: Aug. 6, 2009

(54) Biocompatible Latent Emulsifiers

(75) Inventors: Steven B. Harris, Ontario (CA); Nick Huang, San Bamacino, CA (US)

Correspondence Address: JAY P. HENDRICKSON, 1010 B STREET, SUITE 319, SAN RAFAEL, CA, 94901, US

(73) Assignee: BIOAVAILABILITY, INC., Ranchgo Cucamonga, CA (US)

(21) Appl. No.: 12/084,930

(22) PCT Filed: Nov. 17, 2006
(86) PCT No.: PCT/US2006/044512

§ 371 Date: May 13, 2008

Related U.S. Application Data
(60) Provisional application No. 60/737,871, filed on Nov. 17, 2005.

(51) Int. Cl. A61K 31/122; A61K 31/355

(52) U.S. Cl. 424/94.1; 514/458

(57) ABSTRACT

A category of biocompatible dual-purpose emulsifiers for therapeutic and/or nutritional use is disclosed. The emulsifiers have at least two functions, which depend on pH and surrounding environment. In the anhydrous environment, they serve as nonionic solvents to dissolve the hydrophobic material, such as a hydrophobic drug or nutrient. When in the alkaline aqueous environment of the small intestine, they are transformed into a biocompatible ionic emulsifier. The emulsifiers can also be used to thin the surfactant/hydrophobe mixture in self-emulsifying bases and, thus, accelerate the emulsifying speed of the base without overloading the emulsifying capacity of the emulsion base.

### RELATED APPLICATION

This application relates to and claims the benefit under 35 U.S.C. § 119(e) of U.S. Provisional Application No. 60/737,871 filed Nov. 17, 2005

### FIELD OF THE INVENTION

The present invention relates to compositions that enhance the bioavailability of hydrophobic and lipophilic oral drugs, nutrients and supplements.

### BACKGROUND OF THE INVENTION

In order to increase the bio-availability of hydrophobic material, the common practice is to add sufficient amount of emulsifier. According to the well accepted digestion-absorption theory, if the hydrophobic material is broken into numerous smaller droplets (emulsification), the larger contact surface area will allow better absorption. However, present Food and Drug Administration (FDA) regulations in the United States, as set forth in Title 21 of the Code of Federal Regulations (21 CFR), set maximal allowances for all of the commonly used nonionic food and nutritional emulsifiers. For example, for polysorbate 80 (a PEG ester of sorbitan monooleate), the limits are 0.1 to 0.4% for various food items, and approximately 300 mg/day for nutrients and supplements (See 21 CFR 172.840). This daily legal limit for nutrients and supplements is far below the amount required for sufficient emulsification and dissolution of many hydrophobic substances.

In the case of inexpensive bio-active hydrophobic materials, the relative lack of emulsifier (and sometimes also solvent) may sometimes be compensated by over supply of the hydrophobic blo-active material, which then appears as free oil or crystal. For example, alpha-tocopherol, carotenoids and CoQ10 are commonly supplied in this manner. A notable case is beta-carotene supplement for horses. Under room temperature, beta-carotene is almost insoluble in most of the common organic solvents; therefore, beta-carotene is usually prepared as fine crystals, then this beta-carotene powder is suspended in either vegetable oil, or as water-soluble matrix such as gelatin. Although the serum concentration of beta-carotene can be increased by feeding horses with such insoluble beta-carotene formulas, most of the un-dissolved beta-carotene crystals pass through the G.I. tract without change. This is a good example of a case where a larger amount of biocompatible emulsifier and solvent are needed for good bio-availability of the hydrophobic drug or nutrient or food supplement.

Further, finding a way to reduce the amount of waste and, at the same time, increase bio-availability is particularly important for expensive hydrophobic drugs like, for example, cyclosporin, and for poorly soluble nutrients such as coenzyme Q10 or the rarer carotenoids such as lycopene. In the interest of reducing the cost of beneficial drugs, these high priced materials should not be wasted.

The conventional method used to overcome this poor solubility problem and boost bio-availability requires the utilization of large amounts of hydrophobic solvent to dissolve these expensive hydrophobic materials, and this mixture in turn requires correspondingly large amounts of surfactant to sufficiently emulsify. More specifically, when a nonionic surfactant containing polyethylene glycol (“PEG-containing nonionic surfactant”) is used as the emulsifier, the conventional method requires the use of amounts of surfactant which far exceed the amounts generally considered to be safe for human consumption and, specifically, exceed amounts allowed by 21 CFR in order to achieve sufficient levels of concentration in blood. Although the daily allowance limit is higher for certain digestible ionic surfactants (e.g. lecithin and other phospholipids), these compounds are usually solid and are often poorly suited to making liquid self-emulsifyable preparations.

Surfactants are bi-philic molecules in that they always have a hydrophobic part and a hydrophilic part in the same molecule. Because all surfactant molecules must have a hydrophilic part, they are not hydrophobic enough to dissolve substantial amounts of severely hydrophobic materials. Thus, for larger supplemental doses of bio-active hydrophobes (e.g. alpha-tocopherol, beta-carotene, lycopene, CoQ10, etc), the self-emulsification approach reaches a bottleneck set by the permitted intake of nonionic emulsifier. Since the total amount of PEG-containing nonionic surfactant (e.g. polysorbate-80) is legally controlled at relatively small daily amounts, many of the supplements (e.g. lycopene and CoQ10) utilizing a PEG-containing nonionic surfactant must be prepared as poorly-absorbed solid dispersions of the nutrient.

A nonionic biocompatible and digestible emulsifier is sought to provide enhanced bioavailability of various nutrients and supplements without exceeding the present food and supplement additive regulatory system requirements and is generally considered be safe for human consumption. However, most of the current nonionic surfactants are prepared from polyethylene oxide and may contain a small amount of dioxane; therefore, a formula that mainly depends on a nonionic surfactant may not be suitable for applications that require a large daily dose, such as a nutritional supplement.

The alkaline metal salts of fatty acids (common hard soaps) are the ancestors of all modern emulsifiers; soaps are known from antiquity. However, common soap is less than suitable for oral preparations. Many soaps have high melting points, which means the blending/mixing operation is difficult. More importantly, metallic soaps are also quite poor as pure solvents for hydrophobes. However, when ingested, soaps are transformed to free fatty acids by stomach acid, and then partly disassociated into ionized fatty acid salts again, by the action of basic pancreatic juice. After careful examination of the mechanism of mammalian digestion, the inventors have determined that the free fatty acids can be used, as the carrier medium for hydrophobic materials in oral preparations. Free fatty acids act as much better solvents than do their metal salts in oral liquid preparations, yet they are partly neutralized in the duodenum into soap-like action; thus free fatty acids contribute to emulsification and dispersion of oral preparations in which they are used as lipophilic solvents.

The current invention employs a not previously utilized idea: instead of using soap as an oral emulsifier, it is possible and reasonable to use anhydrous hydrophobic acidic species, including but not limited to biocompatible free fatty acids and their free-acid derivatives. These species are not soaps as ingested, but they eventually act as soaps and natural emulsifiers in the digestive tract. Thus, certain anhydrous biocompatible hydrophobic acids can be used at one time as: 1) solvent, 2) carrier medium and 3) latent emulsifier to carry hydrophobes for oral application.

Fatty acid salts (soaps) have been used from antiquity as emulsifiers, though this use has been in topical, rather than oral, preparations. A great deal of prior technology teaches free fatty acids and lipid soluble carboxylic acids as possible hydrophobic co-solvents in emulsion preparation. However, this technology does not teach free fatty acids as emulsifiers or as pro-emulsifiers. In fact the technology teaches that free fatty acids cannot be used as surfactants. For example, in U.S. Pat. No. 5,952,004 (Rudnic, et al.) the inventors' state:
Further, certain materials, when combined in accordance with the invention to form a water-in-oil microemulsion, give enhanced absorption capabilities. These materials are an oily phase, composed of long chain fatty acids or esters or alcohols thereof, an aqueous phase composed primarily of water, and a surface active agent, primarily of the nonionic block copolymer type, that are mixed together to form a water-in-oil microemulsion.
The long chain carboxylic acids, [sic] generally contain from 4-36 carbon atoms and preferably contains at least 12 carbon atoms, most preferably 12 to 22. In some cases this carbon chain is fully saturated and unbranched, while others contain one or more double bonds. They can have saturated, unsaturated, branched or straight chain hydrocarbon chains. A few contain 3-carbon rings or hydroxyl groups. The compounds are not surface active. They are poorly soluble in water and the longer the acid chain and the fewer the double bonds, the lower the solubility in water. The carboxylic acid group is polar and ionized at neutral pH. This accounts for the slight solubility of short-chain acids in water (italics supplied).

This disclosure and teaching that free fatty acids in emulsion preparations are not “surface active agents”, meaning that the fatty acids do not act as emulsifiers, is typical. Rudnic et al. point out that short chain fatty acids are ionized at neutral pH, giving them a small solubility, and imply that long chain fatty acids are not ionized and, thus, are even less soluble in water. In fact, the pKa of oleic acid has been measured to be as high as 9.85 (Kanicky, 2002), and thus it is essentially water insoluble at neutral pH. Further, medium to long chain (C8 to C22) fatty acids have very limited water solubility; when mixing with water these fatty acids tend to separate from water completely just as oil or fat do.

Thus, what is needed is a therapeutic and/for nutritional composition for oral administration of a unit dosage which does not contain an amount of nonionic surfactants in excess of the legally allowed daily amounts and is generally considered to be safe for human consumption.

### BRIEF SUMMARY OF THE INVENTION

A unit dosage composition for oral administration to a human for therapeutic and/or nutritional use is disclosed which comprises: carboxylic acid and a therapeutic and/or nutritional hydrophobic agent, wherein in a preferred embodiment the carboxylic acid is oleic acid. In another embodiment, a unit dosage composition for oral administration to a human for therapeutic and/or nutritional use is disclosed which comprises: carboxylic acid, a PEG-containing nonionic surfactant, and a therapeutic and/or nutritional hydrophobic agent, wherein the carboxylic acid is again preferably oleic acid.

### DETAILED DESCRIPTION OF THE INVENTION

A therapeutic and/or nutritional unit dosage composition for oral administration is disclosed. The unit dosage composition consists of a free carboxylic acid solvent, preferably free oleic acid, which is lipid-soluble, biocompatible, and in liquid or solid form, and a therapeutic and/or nutritional hydrophobic agent. As used herein, “free carboxylic acid solvent” means a naturally occurring fatty acid of the form R-COOH, where R is an alkane or alkene with 8 to 22 carbons. In general, the composition is prepared by combining the carboxylic acid solvent and hydrophobic agent and heating and stirring the combination until the agent dissolves into the solvent, forming a solution. The solution is allowed to cool and then a portion of the solution is added to a soft gelatin capsule, suitable for oral administration, thereby forming the unit dosage composition. When the capsule containing the solution is ingested by a human being or other mammal, the capsule dissolves, which releases the solution into the digestive tract. When the solution ultimately comes into contact with the aqueous environment of the small intestine, a portion of the carboxylic acid solvent is neutralized and transformed into an ionic surfactant in situ. The ionic surfactant then acts as a pro-surfactant or “latent surfactant,” in that the neutralized carboxylic acid is now able to emulsify the hydrophobic agent, thereby enhancing the agent's bioavailability. As a result, long-chain (C8 or longer) carboxylic acid can be used to replace, in whole or in part, the commonly used nonionic nutritional and pharmaceutical emulsifiers, such as polysorbate 80. In this regard, when carboxylic acid is used to replace a predetermined amount of polysorbate 80, the carboxylic acid portion of the liquid composition will function both as a solvent and a latent emulsifier.

In another embodiment, the use of free carboxylic acid solvent can be supplemented with the use of additional solvents to assist with the formation of a solution of certain extreme hydrophobes, and also with the use of relatively small amounts (such as a few tens of milligrams) of PEG-containing nonionic surfactants, to start the emulsification process in the stomach. In this regard, see Example 03 below which illustrates the use of a PEG-containing nonionic surfactant in a 1 gram capsule.

This new class of biocompatible emulsifiers using carboxylic acid solvents to replace, in whole or in part, PEG-containing nonionic surfactants in delivery systems of therapeutic agents and/or nutritional supplements for oral administration will be extremely useful, since FDA regulations and generally accepted criteria severely limit the amounts of PEG-containing nonionic surfactants that may be added to therapeutic agents and nutritional supplements. However, no similar restrictions are imposed on the use of carboxylic acids in that there are currently no regulatory or generally accepted limitations in the daily allowance of carboxylic acids as additives to either therapeutic agents or nutritional supplements. Therefore, the use of a carboxylic acid as a digestible biocompatible latent emulsifier is a significant improvement in the blo-availability of lipid soluble substances in oral applications.

In our search for biocompatible emulsifier systems, we were surprised to find that carboxylic acids can be used as biocompatible solvents and latent surfactants (pro-surfactants) because these liquid carboxylic acids and their derivatives are fully digestible and completely safe, yet make good solvents for hydrophobes and excellent components for oral self-emulsifying systems. These unique properties (e.g. good hydrophobic solvent, no toxicity, biocompatibility, digestibility, and non-irritative properties) are not found in modern synthetic emulsifiers.

Under room temperature, a lipid soluble liquid carboxylic acid, preferably oleic acid, is used as a liquid solvent. When this acidic species contacts basic small intestine juice, the acid will react with the base and form a biocompatible emulsifier (soap) in situ. This neutralization reaction is sufficient to disperse most of the nutritional supplement products; however, if a small amount of PEG-containing nonionic surfactant (such as polysorbate 80) is added to the mixture, the dissolution process will start faster. The addition of optional small amounts of a PEG-containing nonionic surfactant allows the liquid hydrophobic carboxylic acid to quickly break down to small droplets in the stomach at low pH; these small droplets of carboxylic acid have larger surface area when exposed to basic pancreas juice, leading to more complete ionization of the carboxylic acid. Thus, with the help of small amounts of nonionic emulsifiers (such as PEG esters like polysorbate 80) as initial emulsifiers, one can then use lipid-soluble biocompatible carboxylic acids as latent emulsifiers.

This category of emulsifier is nontoxic and digestible. Before being neutralized by basic small intestine juice, the anhydrous lipid-soluble liquid free fatty acid also serves as solvent/thinner in the solution. By combining both solvent/thinner and emulsifier in one component, a highly bio-available emulsion base composition for therapeutic and/or nutritional use can be prepared with very limited time and resources.

The following examples further describe and illustrate the present invention:

### Example 01

### Alpha Tocopherol, Oleic Acid and Optional Emulsifier

This example demonstrates the use of oleic acid as a solvent and emulsifier with a small amount of traditional nonionic emulsifier (e.g. polysorbate 80) to prepare an oral nutritional supplement product with higher bioavailability.

In a beaker place alpha tocopherol 50 g (50%), with polysorbate 80 (Tween-80) 5 g (5%) and oleic acid 45 g (45%). After sufficient stirring, this mixture forms a clear homogeneous solution, which is suitable for soft gel packing. This tocopherol solution in a soft gel capsule is perfectly suitable for oral nutritional supplement application. It forms an emulsion in basic (pH 8.8) agitated conditions, such as are encountered in the duodenum.

### Example 02

### Alpha Tocopherol and Oleic Acid

Alpha-tocopherol is a viscous liquid; usually it is supplied as pure liquid or co-exists with wheat germ oil. Its high viscosity can be effectively reduced by diluting with oleic acid.

In a beaker place alpha tocopherol 50 g (50%), with oleic acid 50 g (50%). After sufficient stirring, this mixture forms a clear homogeneous low viscosity solution, which is suitable for soft gel packing. This tocopherol solution in a soft gel capsule is perfectly suitable for oral nutritional supplement application with no legal dose limitation in the U.S. It forms an emulsion in basic (pH 8.8) agitated conditions, such as are encountered in the duodenum.

### Example 03

### Coenzyme Q, Solvent, Oleic Acid and Optional Emulsifier

This example demonstrates that oleic acid can be used to replace most of the nonionic emulsifier (e.g. polysorbate 80) in a water-dispersible food supplement preparation, yet retain good oral bioavailability for the supplement substance. Optional second and third hydrophobic solvents (orange oil and ethyl oleate) are used, which are chosen to dissolve coenzyme Q10.

In a beaker place coenzyme Q10 10 g (10%), orange oil 10 g (10%), ethyl oleate 20 g (20%), with a PTFE coated magnetic stirring bar. The mixture is heated to 50° C. with sufficient stirring for solution. At this temperature, coenzyme Q10 is completely dissolved to a transparent orange colored solution. Finally, add Tween-80 5 g (5%) and oleic acid 55 g (55%) to this solution under sufficient stirring, and allow the solution to cool to 30-40° C. After packaging in a soft gel capsule (1 gram fill), this coenzyme Q solution is stable to re-crystallization at room temperature, and is perfectly suitable for oral nutritional supplement application, with permitted labeling allowing use of up to 6 capsules per day (600 mg CoQ10, 300 mg polysorbate). This preparation has superior bioavailability to one which contains the same amount of polysorbate and no oleic acid. It also has superior bioavailability to a preparation which contains only orange oil and ethyl oleate as solvents, and no polysorbate or oleic acid.

### Example 04

### Coenzyme Q, Solvent, and Oleic Acid

This example demonstrates oleic acid can be used as ionic emulsifier in a water-dispersible oral nutrition supplement preparation, yet such formulation retains reasonable oral bioavailability for the supplement substance. Optional second and third hydrophobic solvents are used (orange oil and ethyl oleate), which are chosen to dissolve coenzyme Q10.

In a beaker place coenzyme Q10 10 g (10%), orange oil 10 g (10%), ethyl oleate 20 g (20%), with a PTFE coated magnetic stirring bar. The mixture is heated to 50° C. with sufficient stirring to form solution. At this temperature, coenzyme Q10 is completely dissolved to a transparent orange colored solution. Finally, add oleic acid 60 g (60%) to this solution under sufficient stirring, and allow the solution to cool to 30-40° C. After packaging in a soft gel capsule (1 gram fill), this coenzyme a solution is stable to re-crystallization at room temperature, and is perfectly suitable for oral nutritional supplement application, which is not limited in maximum dose by any components save orange oil, and which can be formulated by replacement of orange oil by ethyl oleate and decrease in coenzyme Q10, into a preparation without legal dose limitations as an oral food supplement, in the United States.

Although the present invention has been described in its preferred embodiment and in certain other embodiments, it will be recognized by those skilled in the art that other embodiments and features may be provided without departing from the scope of the invention, which is defined by the appended claims.

1. A unit dosage composition for oral administration to a human for therapeutic and/or nutritional use, comprising: a monounsaturated free carboxylic acid and a therapeutic and/or nutritional hydrophobic agent, whereby the composition does not include a surfactant.

2. The unit dosage composition of claim 1 in which the carboxylic acid is free oleic acid.

3. The unit dosage composition of claim 1 in which the hydrophobic agent is alpha tocopherol.

7. A unit dosage composition for oral administration to a human for therapeutic and/or nutritional use, comprising: free oleic acid having a concentration of about 50% by weight of the composition and alpha tocopherol having a concentration of about 50% by weight of the composition, whereby the composition does not include a surfactant.

5. (canceled)

6. (canceled)

7. (canceled)

8. (canceled)

9. (canceled)

4. The unit dosage composition as in claim 1 further comprising at least one solvent.

5. The unit dosage composition of claim 4 in which the carboxylic acid is free oleic acid.

6. The unit dosage composition of claim 4 in which the hydrophobic agent is coenzyme Q10.

8. A unit dosage composition for oral administration to a human for therapeutic and/or nutritional use, comprising: free oleic acid having a concentration of about 60% by weight of the composition, coenzyme Q10 having a concentration of about 10% by weight of the composition, orange oil having a concentration of about 10% by weight of the composition, and ethyl oleate having a concentration of about 20% by weight of the composition.

14. (canceled)

15. (canceled)

16. (canceled)

17. (canceled)

9. A unit dosage composition for oral administration to a human for therapeutic and/or nutritional use, comprising: free oleic acid having a concentration of about 55% by weight of the composition, polysorbate 80 having a concentration of about 5% by weight of the composition, coenzyme Q10 having a concentration of about 10% by weight of the composition, orange oil having a concentration of about 10% by weight of the composition, and ethyl oleate having a concentration of about 20% by weight of the composition.
